(19) United States
(12) Patent Application Publication
(10) Pub. No.: US 2016/0109436 A1
(43) Pub. Date: Apr. 21, 2016

(54) SENSOR HAVING CHEMICAL INSPECTING FUNCTION AND THE INSPECTING MATERIAL CONTAINED THEREIN

(71) Applicant: National Tsing Hua University, Hsinchu (TW)

(72) Inventors: Chung-Yao YANG, Hsinchu (TW); Chao-Min CHENG, Hsinchu (TW); Szu-Ting LIN, Hsinchu (TW)

(21) Appl. No.: 14/692,347

(22) Filed: Apr. 21, 2015

(30) Foreign Application Priority Data
Oct. 16, 2014 (TW) 103135895

(51) Int. Cl. G01N 33/52

(52) U.S. Cl. CPC G01N 33/525

(57) ABSTRACT

The present invention provides a sensor having chemical inspecting function and the inspecting material contained therein. In simple terms, one of the major features of the present invention is to reduce the detection area contacted with air between the gel and the inspecting material so as to slow down the degradation thereof. The other feature of the invention is to provide the detailed compositional parameters required by the inspecting material in the said process so as to obtain a balanced value between the cost and the visibility. By the present invention, the problem of short retention period of the personal hygiene item is herein solved.

### CROSS REFERENCE TO RELATED APPLICATION

This application claims priority to Taiwan Patent Document No. 103135895, filed on Oct. 16, 2014 with the Taiwan Patent Office, which is incorporated by reference in its entirety.

1. Field of the invention

The present invention relates to a sensor having chemical inspecting function and the inspecting material contained therein; more particularly, to the sensor with long storage period inspecting the chemicals and the main inspecting material for application.

2. Description of the prior art

The food preservatives keep the food from spoiling by extending the expiration date, and the main advantage of the food preservatives is preventing food poisoning. Therefore, most processed foods comprise food preservatives, and the major food preservatives are sulfite, formaldehyde, borax, sulfur dioxide and nitrate.

In the past, in order to detect the chemical materials mentioned above, people mainly immerse the paper into the liquid inspecting material like oxidase and enzyme to allow the liquid inspecting material to be absorbed evenly by the paper for manufacturing test papers. When applying the conventional test papers, the designated element of the analyte reacts with the inspecting material in the test paper to make the inspecting material in the test paper change color, which then further allows the user to know if the analyte comprises the designated element for detection. Besides, the application for inspecting the chemicals in the body fluid by dropping the inspecting material or mapping on the surface of the sensor such as hygiene products like diapers and sanitary napkins.

The U.S. Pat. No. 6,203,496 relates to a diaper comprised by a permeable and water sucking layer and an impermeable layer having a chemical detection reagent for inspecting the elements in urine with color changing. Besides, the Taiwan Patent M456182 revealed that mapping the pH detection reagent on the side of the impermeable exterior layer of the diapers is able to detect the pH value of the urine.

However, the conventional enzyme test paper has many defects like short storage period, poor heat resistance, large amounts of usage due to bad optical absorption and large amounts of the test papers for the immersion process. More particularly, comparing with the storage period of the conventional hygiene products is on a basis of “year” (for instance, the storage period of the throw-away cotton diapers is approximately five years), the storage period of the enzymes nowadays could be shorter than one hundred days and quickly degrade due to the affect of the temperature, which becomes unable to be commonly used.

The conventional problems above mentioned are the present techniques of the method of the fiber base sensor in the invention.

### SUMMARY OF THE INVENTION

After many tests and experiments, the present invention provides a method and material with simple process and low cost to extend the storage period of the conventional inspecting material like the enzyme to solve the said problem of the inspecting material having fast degradation and short storage period, which is considered to be the content of the present invention.

In summary, the present invention provides an inspecting material having detection function and long storage period, and the main technical feature is to cover the enzyme with gel to reduce the affection of the air and temperature, and further extend the storage period of the inspecting material without affecting the ability of the detection. The elements of the inspecting material and the method of covering gel with the inspecting material is the feature of the present invention.

To summarize, the main point of the present invention is providing the sensor having chemical inspecting function and the inspecting material contained therein and the patch sensor having chemical inspecting function and the inspecting material contained therein. In simple terms, one of the major features of the present invention is to reduce the detection area contacted with air between the gel and the inspecting material so as to slow down the degradation thereof. The other feature of the invention is providing the detailed compositional parameters required by the inspecting material in the said process so as to obtain a balanced value between the cost and the visibility. By the present invention, the problem like short retention period of the personal hygiene item is herein solved.

### BRIEF DESCRIPTION OF THE APPENDED DRAWINGS

Some of the embodiments will be described in detail, with reference to the following figures, wherein like designations denote like members, wherein:

FIG. 1A and FIG. 1B show a schematic diagram of the sensor having chemical inspecting function in the first embodiment of the present invention with different angles.

FIG. 2 shows a schematic diagram of the sensor having chemical inspecting function in the second embodiment of the present invention.

FIG. 3 shows a schematic diagram of the sensor having chemical inspecting function in the third embodiment of the present invention.

FIG. 4 shows a schematic diagram of the forth embodiment of the present invention.

FIG. 5A and FIG. 5B show a schematic diagram of the patch sensor having inspecting function in the first embodiment of the present invention with different angles.

### DETAILED DESCRIPTION OF THE INVENTION

The examples and explanations are mentioned below to well describe the features and spirits of the invention. More importantly, the present invention is not limited to the embodiment described herein. Those skilled in the art will readily observe that numerous modifications and alterations of the device may be made while retaining the teachings of the invention.

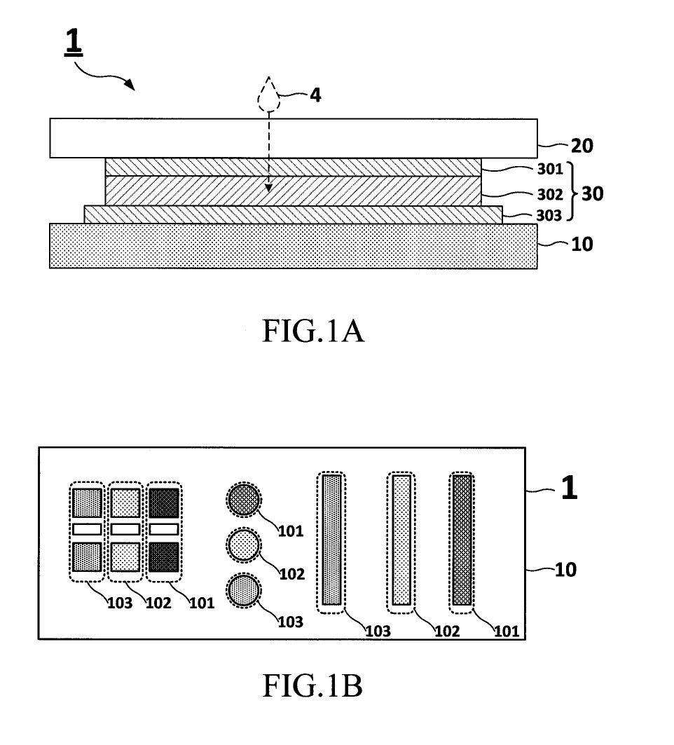

As mentioned before, the present invention provides the sensor having chemical inspecting function and the inspecting material contained therein. Please refer to FIG. 1A to FIG. 1B. FIG. 1A and FIG. 1B show a schematic diagram of the sensor having chemical inspecting function in the first embodiment of the present invention with different angles. Firstly, the sensor having chemical inspecting function of the present invention comprises the first carrier 10 and the inspecting material 30. In the embodiment, the first carrier 10 is a gel and a water-insoluble polymer material or a water-soluble polymer material. The water-insoluble polymer material mentioned above is a material with low moisture absorption like polymer fiber, and the water-soluble polymer material is a material with high moisture absorption like cotton.

Besides, the inspecting material 30 comprises the gel 31 and the reacting substance 32 covered by the gel 31 to reduce the contact area between the reacting substance 32 and the air wherein the analyte 4 penetrates the gel 31 to react with the reacting substance 32 for changing the color of the reacting substance 32 when the analyte 4 is contacted with the inspecting material 30.

With regard to the material of the gel 31 and the reacting substance 32, the material of the gel 31 is a polymer material. More particularly, the polymer material mentioned above is polyvinyl alcohol, PVA, not limited to the embodiment described herein can be replaced by other transparent water-soluble material.

Otherwise, as to the different analyte 4, the reacting substance 32 of the present invention can be the nitrite detection reagent, the glucose detection reagent, the human serum albumin (HSA) detection reagent, the bilirubin detection reagent, the pH detection reagent, the ketone body detection reagent, the urobilinogen detection reagent and other substances capable of color change while reacting with relative chemicals.

Besides the gel 31 and the reacting substance 32 mentioned above, the user can add other additives to get other features in the application. More importantly, the inspecting material 30 formed by the gel 31 and the reacting substance 32 can be formed and applied by different methods. The application of the inspecting material 30 will be illustrated below.

Please refer to the FIG. 1A to FIG. 1B, in the embodiment, the sensor 1 having the inspecting function of the present invention comprises the first carrier 10, the surface of the first carrier 10 comprising the inspecting area 101, the inspecting area 102 and the inspecting area 103 set the inspecting material 30 of the present invention. In the embodiment, the inspecting material 30 comprises the lower gel layer 303, the upper gel layer 301 and the inspecting layer 302 set between the upper gel layer 301 and the lower gel layer 303 contacted with the first carrier 10. The lower gel layer 303 and the upper gel layer 301 comprise the gel 31 separately. The inspecting layer 302 comprises the reacting substance 32. In the embodiment, the upper gel layer 301 and the lower gel layer 303 are formed by the water-soluble polymer material like PVA.

In real application, the analyte 4 like the urine can be transmitted by the second carrier 20 set on the upper gel layer 301 and contacted with the upper gel layer 301 of the inspecting material 30, the water of the analyte 4 is contacted with the reacting substance 32 of the inspecting layer 302 by melting the upper gel layer 301 to change the color. According to actual needs, the analyte 4 is not limited to the second carrier 20 but should be contacted with the upper gel layer 301 to change color directly in the present invention.

The structure mentioned above is completed by the steps mentioned below. At first, prepare the liquid gel 31 and the liquid reacting substance 32. In the embodiment, the liquid gel 31 is formed by mixing the particle polyvinyl alcohol (polymerization degree 70000 to 100000) and water to a thicky phase under a specific proportion, and the viscosity is about 8000 to 20000 CPS. It is noted that to the thickness and the viscosity of the polyvinyl alcohol are not limited by the state mentioned above. The thickness and the viscosity can be controlled by controlling the ratio of the polyvinyl alcohol and water.

After preparing the gel 31 and the reacting substance 32, mix a part of the gel 31 with the reacting substance 32 to make a slurry with the reacting substance 32.

In the embodiment, if the analyte 4 is the nitrite, the reacting substance 32 (reacting substance 32 is comprised of 50 mM sulfanilamide, 330 mM citric acid and 10 mM N-(1-naphthyl)ethylenediamine) inspected by the nitrite is mixed with the gel 31 to cover the reacting substance inspected by the nitrite in the inspecting material 30, and the proportion is between 20% to 66%, wherein the efficiency is the best when the proportion is under 29%. The detectable molar concentration of the analyte 4 comprised the nitrite is between 0.1 mM and 5 mM. While the detectable molar concentration falls within the above-mentioned interval, the user can detect the change of the color without wasting too much material. The efficiency mentioned above is the ability of the observation of the user and the amount of the wasted material.

Similarly, if the analyte 4 is the glucose, the reacting substance 32 comprising 75 U/mL glucose oxidase, 15 U/mL Horseradish peroxidase and 0.6 M potassium iodide inspected by the glucose is mixed with the gel 31 to cover the reacting substance inspected by the glucose in the inspecting material 30, and the proportion is between 10% to 50%, wherein the efficiency is the best when the proportion is under 20%. The detectable molar concentration of the analyte 4 comprised of the glucose is between 5 mM and 500 mM, yet the concentration falling the interval from 5 mM to 50 mM is easier to be inspected accurately.

Otherwise, if the analyte 4 is the human serum albumin, the reacting substance 32 (reacting substance 32 is comprised of 250 mM citric acid and 3.9 mM

Horseradish tetrabromophenol blue) inspected by the human serum albumin is mixed with the gel 31 to cover the reacting substance inspected by the human serum albumin in the inspecting material 30, and the proportion is between 20% to 50%, wherein the efficiency is the best when the proportion is under 33%. The detectable molar concentration of the analyte 4 comprised of the human serum albumin is between 2 μM and 1000 μM, yet the concentration falling the interval from 10 μM to 150 μM is easier to be inspected accurately.

If the analyte 4 is the bilirubin, the reacting substance 32 comprising 4.9 mM sodium nitrite, 45 mM sulfanilic acid and 104 mM hydrochloric acid inspected by the bilirubin is mixed with the gel 31 to cover the reacting substance inspected by the bilirubin in the inspecting material 30, and the proportion is between 10% to 50%, wherein the efficiency is the best when the proportion is under 20%. The detectable molar concentration of the analyte 4 comprised of the bilirubin is between 10 mg/mL and 100 mg/mL, yet the concentration falling the interval from 25 mg/mL to 50 mg/mL is easier to be inspected accurately.

If the analyte 4 is the urobilinogen, the reacting substance 32 comprising 0.1 M 4-Dimethylaminobenzaldehyde, and 2.8 M hydrochloric acid inspected by the urobilinogen is mixed with the gel 31 to cover the reacting substance inspected by the urobilinogen in the inspecting material 30, and the proportion is between 16% to 50%, wherein the efficiency is the best when the proportion is under 33%. The detectable molar concentration of the analyte 4 comprised of the urobilinogen is between 17 μM and 500 μM, the concentration falling the interval from 20 μM to 200 μM is easier to be inspected accurately.

If the analyte 4 is the ketone body, the reacting substance 32 comprising 3% sodium nitroprusside, and 0.2 M glycine inspected by the ketone body is mixed with the gel 31 to cover the reacting substance inspected by the ketone body in the inspecting material 30, and the proportion is between 20% to 66%, wherein the efficiency is the best when the proportion is under 50%. The detectable molar concentration of the analyte 4 comprised of the ketone body is between 0.5 mM and 200 mM, yet the concentration falling the interval from 5 mM to 200 mM is easier to be inspected accurately.

While inspecting the pH of the analyte 4, the reacting substance 32 comprising universal indicator inspected by the pH is mixed with the gel 31 to cover the reacting substance inspected by the pH in the inspecting material 30. The proportion is between 16% and 50%, wherein the efficiency is the best when the proportion is under 20%. The detectable molar concentration of the analyte 4 comprised of the pH is between pH 4.0 and pH 10.0.

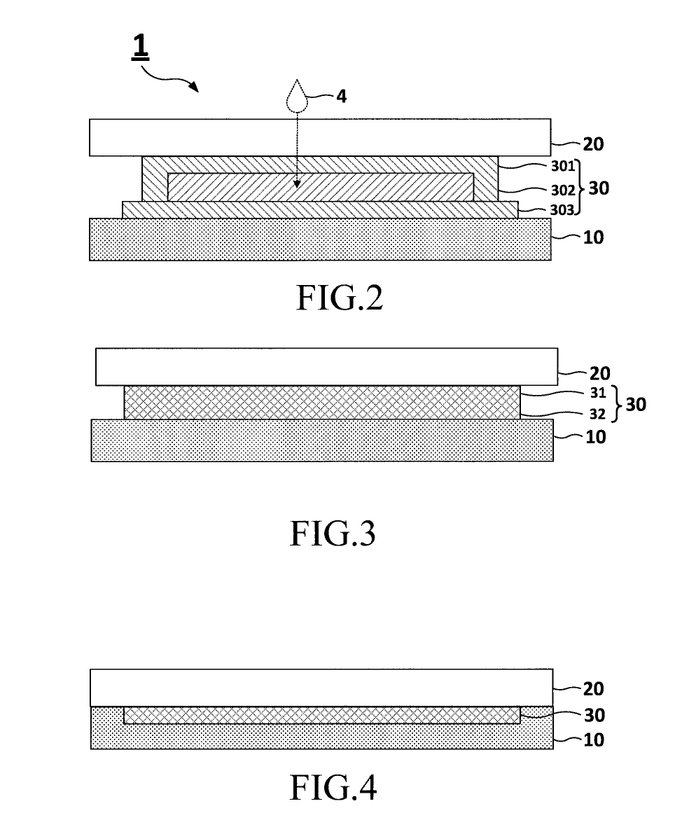

The gel 31 and the slurry are set into a shaping device. The shaping device mentioned above is the device with the stamping process, the transferring process, the dispensing process or the screen printing process. After preparing the device, form the lower gel layer 303 on the surface of the first carrier 10 by the gel 31, then form the inspecting layer 302 on the lower gel layer 303 by the slurry, and finally form the upper gel layer 301 on the inspecting layer 302 by the gel 3, which acquires the structure of the FIG. 1A. More importantly, the upper gel layer 301 needs to connect with the lower gel layer 303 on the side of the inspecting layer 302 to cover the inspecting layer 302 as illustrated in the second embodiment of the FIG. 2. After forming the structure, drying the water and getting hardened, the process is complete. While proceeding with the steps of forming the materials mentioned above, if the material gets thinner, the material needs to cooperate with the adjustment of the temperature to harden the parts or all of the material and set the surface of the material on the surface to get a closer structure.

Except for the design mentioned above, please refer to the FIG. 3. FIG. 3 shows a schematic diagram of the sensor having chemical inspecting function in the third embodiment of the present invention. The difference from the previous embodiment is that the inspecting material 30 is a single-layer. More particularly, the design of the upper gel layer 301, the inspecting layer 302 and the lower gel layer 303 mentioned above is different; the inspecting material 30 of the embodiment is coated with the slurry mentioned above on the surface of the inspecting area to be completed, which skips the applications of the upper gel layer 301 and the lower gel layer 303. The method of forming the structure of the embodiment is similar to the previous embodiment, which is not detailed again herein.

Moreover, except for the structure mentioned above, the present invention comprises the forth embodiment. The material is similar to that of the third embodiment, but the thickness is lower enough to penetrate into the surface of the sensor 1. The difference between the embodiment and the previous embodiment is the concentration and the thickness of the gel 31 and the reacting substance 32 of the slurry. By adding water into the slurry, the slurry gets a better fluidity to penetrate into the void of the fiber. After the slurry penetrated into the fiber and the water of the gel vaporized, the gel 31 and the reacting substance 31 is formed on the surface and the interior of the fiber evenly.

Otherwise, please refer to the design of the first embodiment of the present invention. The present invention provides the patch sensor having inspecting function. Please refer to FIG. 5A and FIG. 5B. FIG. 5A and FIG. 5B show a schematic diagram of the patch sensor having inspecting function in the first embodiment of the present invention with different angles. The structure design of the patch sensor 2 having inspecting function of the present invention is similar to the sensor having inspecting function of the first embodiment of the present invention. However, the difference is that the first carrier 10 is able to be set with an attached film 40, wherein the first carrier 10 is comprised of the water-insoluble material and the water-soluble material; the inspecting material is comprised of the water-insoluble material; the first carrier 10 is comprised of first surface S1 and the second surface S2; the inspecting material 30 is set on the first surface S1; the attached film 40 is set on the upper surface S3 with an adhesive substance; and the attached film 40 is adhered to the second surface S2 of the first carrier 10 by the upper surface S3.

When the patch sensor 2 is in use, the user tears the attached film 40 off and adheres the patch sensor 2 to the article with the analyte 4 to make the inspecting material 30 contact with the analyte 4, which allows the analyte 4 to penetrate into the gel 31 to react with reacting substance 32 to change the color of the reacting substance 32 for inspection. The patch sensor of the present invention is not limited to the above mentioned; users can replace the attached film 40 with upper gel layer 301 having adhesive function according to actual needs. When the patch sensor 2 is in use, the user can utilize the upper gel layer 301 to adhere to the article with the analyte 4.

Additionally, in the embodiment mentioned above, the user can utilize the dispenser of the glue dispensing machines or other methods to form the inspecting material 30 on the surface of the sensor 1 and the patch sensor 2; the part comprised of the inspecting material 30 is the so-called inspecting area. When the sensor 1 and the patch sensor 2 have different types of inspecting material 30, each of inspecting material 30 can be interpreted as the first inspecting material 101, the second inspecting material 20, the third inspecting material 30 and so forth, respectively. Please note that every inspecting area is not limited to a single point but a band, an entire area or any shapes according to users' needs such as the different shapes of the inspecting area showed in the figures. The sensor is able to do the inspection after the inspecting material 30 becomes dry.

On the other hand, the sensor 1 and the patch sensor 2 are able to be applied on personal hygiene items to inspect the physical status of the users. Please refer to the FIG. 1. When the sensor 1 of the present invention is a personal hygiene item like diapers, the outer surface of the film and the hygroscopic cotton is regarded as the first carrier 10 and the second carrier 20 mentioned above.

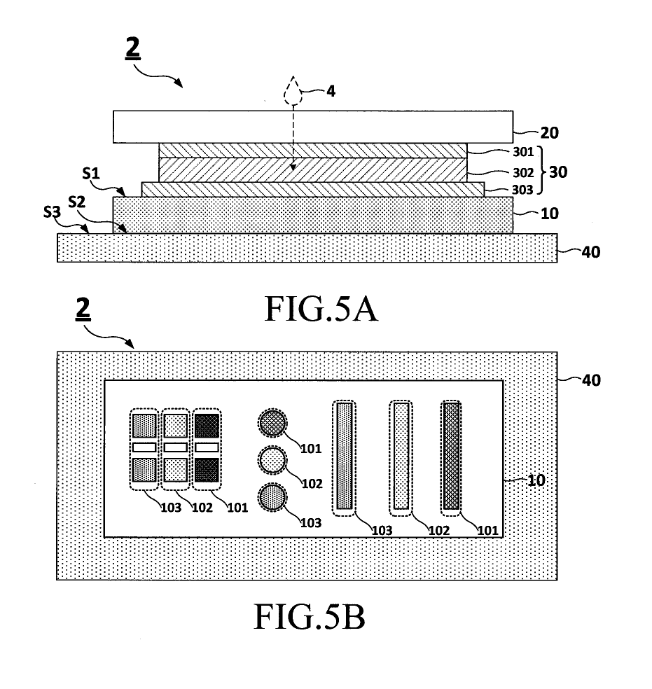

Moreover, please refer to the FIG. 3 and FIG. 4, FIG. 4 is the schematic diagram of the forth embodiment of the present invention. The difference between the forth embodiment and the embodiment showed in the FIG. 3 is that every inspecting area set at least one notch to place the inspecting material 30 inside. By this way, the position of the inspecting material 30 could be set stably and become uneasy to affected by the second carrier 20 illustrated in the design of present invention of the FIG. 4.

To summarize, the present invention provides the sensor having chemical inspecting function and the inspecting material contained therein and the patch sensor having chemical inspecting function and the inspecting material contained therein. In simple terms, one of the features of the present invention is to reduce the detection area contacted with air between the gel and the inspecting material like the enzyme so as to slow down the degradation thereof. The other feature of the invention is to provide the detailed compositional parameters required by the inspecting material in the said process so as to obtain a balanced value between the cost and the visibility. By the present invention, the problem of short retention period of the personal hygiene item is herein solved.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the meets and bounds of the appended claims.

What is claimed is:

1. An inspecting material, comprising at least one gel and a reacting substance, the gel for covering the reacting substance to reduce a contact area between air and the reacting substance, wherein an analyte penetrates the gel to react with the reacting substance for changing the color of the reacting substance when the analyte is contacted with the inspecting material.

2. The inspecting material of claim 1, wherein the gel is a water-insoluble polymer material with at least one series of holes penetrated by the analyte for making the analyte to be contacted with the reacting substance.

3. The inspecting material of claim 1, wherein the gel is a water-soluble polymer material compatible with the analyte penetrating the gel by melting the gel to contact with the reacting substance.

4. The inspecting material of claim 1, wherein the reacting substance is a nitrite detection reagent comprised in the inspecting material, the proportion of the nitrite detection reagent comprised in the inspecting material is from 20% to 66%, wherein the efficiency is the best when the proportion is under 29%, and the detectable molar concentration of the analyze of the reacting substance is between 0.1 mM and 5 mM.

5. The inspecting material of claim 1, wherein the reacting substance is a glucose detection reagent comprised in the inspecting material, the proportion of the glucose detection reagent comprised in the inspecting material is from 10% to 50%, wherein the efficiency is the best when the proportion is under 20%, and the detectable molar concentration of the analyze of the reacting substance is between 5 mM and 500 mM.

6. The inspecting material of claim 1, wherein the reacting substance is a human serum albumin (HSA) detection reagent comprised in the inspecting material, the proportion of the human serum albumin (HSA) detection reagent comprised in the inspecting material is from 20% to 50%, wherein the efficiency is the best when the proportion is under 33%, and the detectable molar concentration of the analyze of the reacting substance is between 2 μM and 500 μM.

7. The inspecting material of claim 1, wherein the reacting substance is a bilirubin detection reagent comprised in the inspecting material, the proportion of the bilirubin detection reagent comprised in the inspecting material is from 10% to 50%, wherein the efficiency is the best when the proportion is under 20%, and the detectable concentration of the analyze of the reacting substance is between 10 mg/mL and 100 mg/mL.

8. The inspecting material of claim 1, wherein the reacting substance is an urobilinogen detection reagent comprised in the inspecting material, the proportion of the urobilinogen detection reagent comprised in the inspecting material is from 16% to 50%, wherein the efficiency is the best when the proportion is under 33%, and the detectable concentration of the analyze of the reacting substance is between 17 μM and 500 μM.

9. The inspecting material of claim 1, wherein the reacting substance is a ketone body detection reagent comprised in the inspecting material, the proportion of the ketone body detection reagent comprised in the inspecting material is from 20% to 66%, wherein the efficiency is the best when the proportion is under 50%, and the detectable concentration of the analyze of the reacting substance is between 0.5 mM and 200 mM.

10. The inspecting material of claim 1, wherein the reacting substance is a pH detection reagent comprised in the inspecting material, the proportion of the pH detection reagent comprised in the inspecting material is 16% to 50%, wherein the efficiency is the best when the proportion is under 20%, and the detectable pH value of the analyze of the reacting substance is between pH 4.0 and pH 10.0.

11. A sensor with an inspecting function, comprising:
a first carrier, having at least one inspecting area, a surface of the inspecting area disposed by the inspecting material of claim 1 to claim 10.

12. The sensor of claim 11, wherein the inspecting material comprises a lower gel layer, an upper gel layer, and an inspecting layer set between the upper gel layer and the lower gel layer, the lower gel layer contacted with the first carrier, the upper gel layer and the lower gel layer comprising the gel respectively, and the inspecting layer comprising the reacting substance.

13. The sensor of claim 12, wherein the inspecting material is formed by compiling the upper gel layer, the lower gel layer, and the inspecting layer.

14. The sensor of claim 11, wherein the inspecting material is doped on the surface of the inspecting area of the inspecting layer.

15. A patch sensor with a detection function, comprising:
a first carrier, having a first surface and a second surface, the first surface having at least one inspecting area, the surface of the inspecting area disposed by the inspecting material of claim 1 to claim 10; and
an attached film, having an upper surface with an adhesive material attached to the second surface of the first carrier.

16. The patch sensor of claim 15, wherein when the patch sensor is in use, the user can tear the attached film and make the patch sensor attach to a substance with the analyte to make the inspecting material on the first carrier contact with the analyte, the analyte penetrates into the gel to react with the reacting substance and makes the color of the reacting substance change to get the efficiency of the detection when the inspecting material is contacted with the analyte.

17. The patch sensor of claim 15, wherein the first carrier comprises a hydrophilic material and a hydrophobic material, and the inspecting area is made by the hydrophilic material.

18. The patch sensor of claim 15, wherein the inspecting material comprises a lower gel layer, an inspecting layer, and an upper gel layer, the inspecting layer is set between the upper gel layer and lower gel layer, the lower gel layer is contacted with the first carrier, the lower gel layer and the upper gel layer comprise the gel respectively, and the inspecting layer comprises the reacting substance.

19. The patch sensor of claim 17, wherein the first inspecting material is formed by compiling the upper gel layer, the inspecting layer, and the lower gel layer.

20. The patch sensor of claim 17, wherein the upper gel layer of the first inspecting layer has viscosity for attaching to the substance with the analyte.
